(19) United States
(12) Patent Application Publication
(10) Pub. No.: US 2024/0115370 A1
(43) Pub. Date: Apr. 11, 2024

(54) PROSTHETIC IMPLANT DELIVERY DEVICE

(71) Applicant: Jeffrey Weinzweig, Highland Park, IL (US)

(72) Inventor: Jeffrey Weinzweig, Highland Park, IL (US)

(21) Appl. No.: 17/960,969

(22) Filed: Oct. 6, 2022

(51) Int. Cl. A61F 2/00

(52) U.S. Cl. CPC A61F 2/0095; A61F 2230/0054; A61F 2250/0039

(57) ABSTRACT

A prosthetic implant transfer device is provided that may be used to insert a prosthetic implant through a surgical opening. In some embodiments, the device may comprise a flexible sheet of material that may be configured to deliver the implant. The flexible sheet of material includes a plurality of securement members, and may enable selection of a desired size distal opening.

### FIELD OF THE INVENTION

This invention is directed to a delivery apparatus for facilitating the insertion of a prosthesis, such as a breast implant, into a surgical pocket, and method for using the device.

### BACKGROUND OF THE INVENTION

This invention relates to a device to facilitate the placement of prosthetic implants within a patient's body. Prosthetic implants, such as breast implants, pectoral implants, gluteal implants, and others have been used extensively for many years. Prosthetic implants can take many forms, many of which are comprised of a compressible or deformable material, such as silicone. In order to place such an implant within a body or a cavity of a patient, an incision is made to access the desired site of placement and develop the pocket or space between the tissue planes where the implant will reside.

In order to reduce trauma to the patient and lessen the risk of infection, it is desirable that the incision in the patient for the surgical pocket be as small as possible. In particular, the incision in the patient may be smaller than the implant itself. Traditionally, a medical professional would manipulate the implant by hand to deform or compress it and guide it through the incision into the surgical pocket. Such direct handling of the implant has several shortcomings. The time involved in manipulating the implant to guide it through the relatively small incision is greater than with a large incision. This additional surgical time increases the risks of infection and complications from anesthesia. The amount of time required also impacts the costs of the procedure because of the additional time of the physician and staff. In some instances, implant failures in the form of minor damage to the outer surface of the implant can also be caused by the manipulation. In addition manual manipulation of the implant may result in mishandling and dropping of the implant, which contaminates the implant, rendering it unusable.

In order to address these problems, one device that has been developed is a flexible tapered funnel to aid in sliding a breast implant through the incision. U.S. Pat. No. 8,211,173 issued to Keller et al. discloses an apparatus and process for delivering a silicone breast implant into a surgical pocket. U.S. Pat. No. 8,211,173 discloses a tapered sleeve having a wider first end for receiving a breast implant, and a smaller second end for insertion into the surgical pocket. By placing the breast implant in the sleeve, a surgeon squeezes the sleeve to deform the breast implant and force it through the second end and into the surgical cavity. In order to accommodate different size implants, the second end of the sleeve is trimmed to create the desired size hole. Transferring of the implant through the sleeve is aided by lubrication of the implant and/or interior surface of the sleeve itself.

While the flexible funnel has provided some advantages, the present invention addresses certain shortcomings to the funnel. Because adjustment of the opening through which the implant is squeezed is done by way of cutting a portion of the end of the sleeve, an error in sizing may result in having to discard the funnel and trim a new one, which can add to the cost of the procedure. In addition to traumatizing the surgical site, the damaged implant cannot be used, which again increases costs. Another problem that has been experienced results when the force required to transfer the implant through the end of the sleeve causes the sleeve itself to burst at a seam. In some instances, the sleeve may fail due to a tear or inadvertent cut at the end of the sleeve accidentally made when the sleeve is trimmed. Another issue that may occur stems from the open proximal end that receives the implant. Because of the substantial lubrication that is utilized to allow the implant to be transferred through the distal end, possible mishandling of the flannel may result in the implant sliding out of the proximal open end.

One approach to addressing issues with an open proximal end are discussed in U.S. Pat. No. 10,105,213. To improve the fabrication of devices, it is desired to provide a device that is configurable by the medical professional to have a closed proximal end.

The present invention seeks to address these shortcomings and provide an improved device.

### SUMMARY OF THE INVENTION

The present invention is directed to a device for inserting a prosthetic implant through a surgical opening. In some embodiments, the prosthetic implant delivery device comprises a flexible sheet of sterilizable semitransparent or transparent material. The sheet preferably has a proximal end portion, a distal end portion, a first side portion, and a second side portion. In a preferred embodiment, the material is a PVC film or vinyl film.

The first side portion preferably includes a side securement member adapted to secure the first side portion with the second side portion so as to connect the first side portion to the second side portion and to form a distal opening in the distal end portion. In other preferred embodiments, the second side portion also includes a securement member which is adapted to secure with the side securement member of the first side portion. The securement members may comprise different embodiments, including, an adhesive, a hook and loop enclosure member, a strip of molded hooks, a lip and groove relationship, and the like. An example of a preferred securement members include 3M Dual Lock Reclosable Fasteners. In other preferred embodiments, the second side securement member may comprise a plurality of attachment portions whereby a distal opening size is selected according to securing the side securement member with one of the plurality of attachment portions.

The proximal end portion of the sheet may include a first transverse portion and a second transverse portion, where the first transverse portion includes a proximal securement member adapted to secure the first transverse portion with the second transverse portion so as to connect the first transverse portion to the second transverse portion. In some embodiments, the first transverse portion may define a flap that is foldable over the second transverse portion. In some preferred embodiments, the second transverse portion may include a second proximal securement member adapted to secure with the proximal securement member of the first transverse portion. In some preferred embodiments, the proximal end portion is fully closed when the first transverse portion is secured with the second transverse portion, whereas in other embodiments, it is partially closed. Preferably, the proximal end is closed to the extent that any opening is smaller than the prosthetic implant such that it is prevented from accidentally exiting out of the proximal end.

### BRIEF DESCRIPTION OF THE DRAWINGS

A fully enabling disclosure of the present invention, including the best mode thereof to one of ordinary skill in the art, is set forth more particularly in the remainder of the specification, including reference to the accompanying drawings.

FIG. 1 is a front perspective view of a preferred embodiment of the present invention in a condition to be configured for use;

FIG. 2 is a rear perspective view of the embodiment of FIG. 1

FIGS. 3A and 3B are a perspective with of the embodiment of FIG. 1, with details related to a preferred configuration for securement members;

FIG. 4 is a perspective view of the embodiment of FIG. 1 with exemplary example of configuring device for use;

FIG. 5 is a perspective view of a preferred embodiment of the present invention configured for use; and

FIG. 6 is an enlarged perspective view of the present invention in use.

### DESCRIPTION OF THE PREFERRED EMBODIMENTS

Reference will now be made in detail to preferred embodiments of the invention, one or more examples of which are set forth below. Each example is provided by way of explanation of the invention, not limitation of the invention. It will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

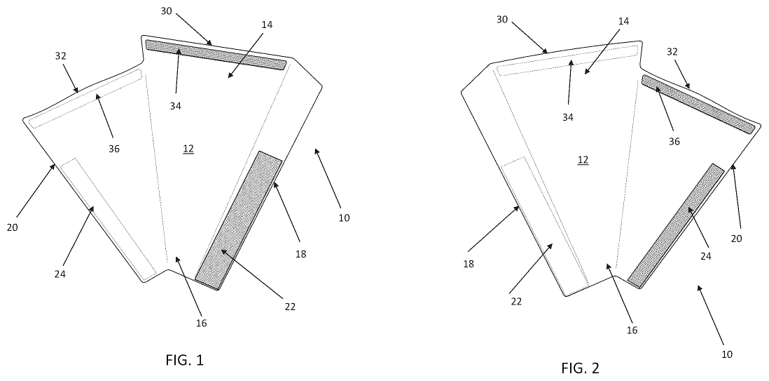

Referring to FIGS. 1 and 2 a preferred embodiment of the present invention is depicted in a condition to be configured for use. For each of manufacturing, this is the condition in which the device is intended to be provided to users. As shown, prosthetic implant device 10 comprises a sheet of material 12 defining a proximal end or end portion 14 and a distal end or distal end portion 16. The flexible sheet 12 is preferably semitransparent or fully transparent such that a user can view the prosthetic implant placed within the device. The sheet 12 is preferably made from a sterilizable material. Various material that may be suitable include Mylar®, plastics made from Tygon® brand of plastics, vinyls, polyvinyl chloride, and other similar materials. The material must also be able to be sterilized by way of conventional sterilization techniques. There are numerous materials that may be suitable, as will be understood by persons skilled in the art.

The sheet 12 also defines a first side portion 18, and a second side portion 20. It should be understood that description of the portions as first or second are only for purposes of reference. In this preferred embodiment, formed with a first side portion 18 is a securement member 22. In this preferred embodiment, second side portion 20 also includes a securement member 24. It is contemplated that in some embodiments, only the first side portion 18 may include a securement member. For example, first side portion 18 may include a securement member comprising an adhesive strip that is exposed by removing a release tape (not shown) and securing the first side portion 18 to the second side portion 20 by pressing the adhesive strip to the second side portion. In the preferred embodiment shown, the first side securement member 22 and the second side securement member 24 cooperate with one another and secure to one another. For example, the first and second securement members may comprise a hook and loop arrangement, cooperating molded hooks, or a lip and groove arrangement.

It is preferred that the second securement member 24 comprises a plurality of attachment portions such as portions 26 and 28. For example, in an embodiment wherein first securement member is a strip of flexible hooks and securement member is a strip of loops, the plurality of attachment portions enable the size of the distal opening, discussed below, to be selectively formed. In an embodiment where a lip and groove arrangement is utilized, a series of grooves may be provided to define the attachment portions.

The proximal end portion 14 preferably includes a first transverse portion 30 and a second transverse portion 32. As will be discussed in further detail below, the first transverse portion preferably includes a securement member 34, which may take the form of the securement members discussed above with respect to the side securement members. In a preferred embodiment, the first transverse portion 30 defines a flap that may be folded over the second transverse portion 32 as part of the securing of the first transverse portion 30 with the second transverse portion 32. In some embodiments, the second transverse portion 32 may also include a securement member 36 that is adapted to secure with securement member 34.

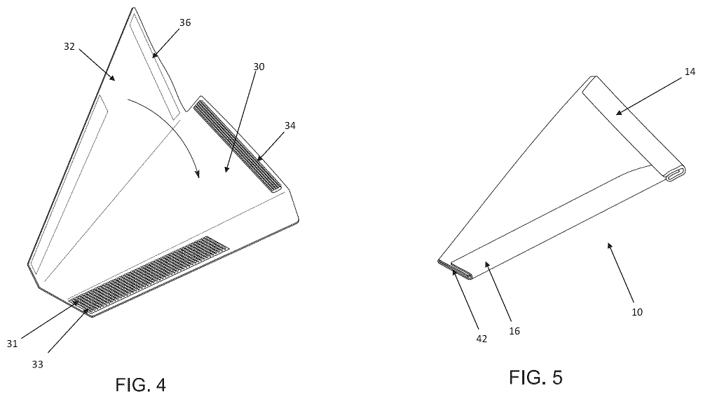

As shown in FIG. 4, in order to configure the device 10 for use, first side portion 18 and second side portion 20 are brought together and securement member 22 and securement member 24 engaged with one another. In a preferred form, the securement members comprises different portions, such as transverse portions 31 and 33 to selectively configure the size of the opening in the distal end. First transverse portion 30 is also folded over the second transverse portion 32, and securement member 34 is engaged with securement member 36.

Referring to FIG. 5, the device 10 is depicted in a configuration adapted to facility insertion of a prosthetic implant (not shown) in a patient. As shown, first side portion 18 of sheet 12 is secured to attachment portion 28 of second side portion 20 (See FIG. 1). Distal opening 42 is therefore defined in the distal end portion 16. When first transverse portion 30 is secured with second transverse portion 32, the proximal end 14 is closed at least partially, but as shown in the preferred embodiment of FIG. 5, is closed fully.

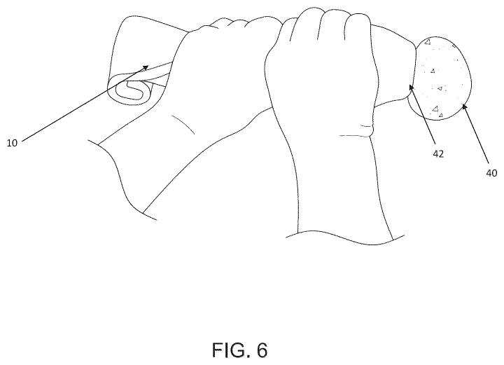

Referring to FIG. 6, a prosthetic implant 40 is placed within the formed prosthetic implant device 10. The surgeon applies a force to the device 10 to transfer the implant 40 distally through the distal opening 42. During a procedure, the distal end of the device 10 is typically inserted 5-10 mm within an incision as the implant 40 is transferred through the incision into a pocket that had been created by the surgeon to receive it. In this particular example, the incision is made in the inframammary fold beneath the breast, however, the incision may also be made through any incision, such as transaxillary and periareolar. This ensures “touchless” and uninterrupted passage and transfer of the implant 40 through the implant device 10 into the surgical pocket while minimizing risk of inadvertent external delivery of the implant 40 or direct contact of the implant 40 with the skin.

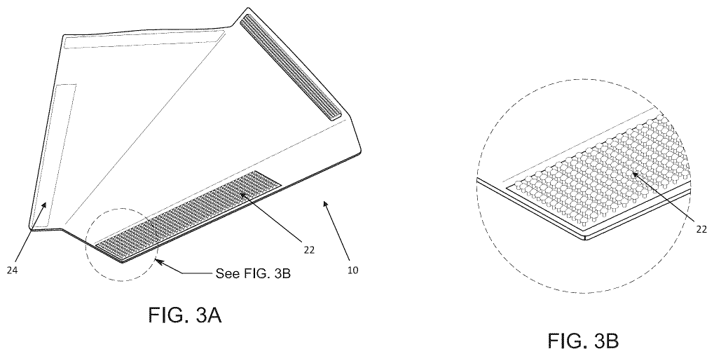

As discussed, the securement members can take different forms. One such form is a hook and loop arrangement. A branded version of such an arrangement is Velcro©. In a preferred embodiment, as shown in FIGS. 3a and 3b, the first side securement member and second side securement member 22 and 24 comprise molded hooks, which in this embodiment resemble mushrooms. The interaction of the molded hooks secure members 22 and 24 together.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention which is set forth in the following claims. In addition, it should be understood that aspects of the various embodiments may be interchanged, both in whole, or in part. Therefore, the spirit and scope of the appended claims should not be limited to the description of the preferred versions contained therein.

What is claimed is:

1. A prosthetic implant delivery device comprising:
a flexible sheet of sterilizable semitransparent or transparent material, the sheet comprising a proximal end portion, a distal end portion, a first side portion, and a second side portion;
the first side portion comprising a side securement member adapted to secure the first side portion with the second side portion so as to connect the first side portion to the second side portion and to form a distal opening in the distal end portion;
the proximal end portion comprising a first transverse portion and a second transverse portion, the first transverse portion comprising a proximal securement member adapted to secure the first transverse portion with the second transverse portion so as to connect the first transverse portion to the second transverse portion.

2. The prosthetic implant device of claim 1, wherein the second side portion comprises a second side securement member adapted to secure with the side securement member of the first side portion.

3. The prosthetic implant device of claim 2, wherein the wherein the side securement member and the second securement member comprise one of a hook and loop enclosure or molded hooks.

4. The prosthetic implant device of claim 2, wherein the second side securement member comprises a plurality of attachment portions whereby a distal opening size is selected according to securing the side securement member with one of the plurality of attachment portions.

5. The prosthetic implant device of claim 1, wherein the second transverse portion comprises a second proximal securement member adapted to secure with the proximal securement member of the first transverse portion.

6. The prosthetic implant device of claim 4, wherein the wherein the proximal securement member and the second proximal securement member comprise one of a hook and loop enclosure or molded hooks.

7. The prosthetic implant device of claim 1, wherein the proximal end portion is closed when the first transverse portion is secured with the second transverse portion.

8. The prosthetic implant device of claim 1, wherein the first transverse portion defines a flap foldable over the second transverse portion.

9. A prosthetic implant delivery device comprising:
a flexible sheet of sterilizable semitransparent or transparent material, the sheet comprising a proximal end portion, a distal end portion, a first side portion, and a second side portion;
the first side portion comprising a first side securement member and the second side portion comprising a second side securement member, the first side securement member adapted to secure the first side portion with the second side portion so as to connect the first side portion to the second side portion and to form a distal opening in the distal end portion and selective the size of the distal opening;
the proximal end portion comprising a first transverse portion and a second transverse portion, the first transverse portion comprising a proximal securement member adapted to secure the first transverse portion with the second transverse portion so as to connect the first transverse portion to the second transverse portion.

10. The prosthetic implant device of claim 9, wherein the wherein the side securement member and the second securement member comprise one of a hook and loop enclosure or molded hooks.

11. The prosthetic implant device of claim 9, wherein the second side securement member comprises a plurality of attachment portions whereby a distal opening size is selected according to securing the side securement member with one of the plurality of attachment portions.

12. The prosthetic implant device of claim 9, wherein the second transverse portion comprises a second proximal securement member adapted to secure with the proximal securement member of the first transverse portion.

13. The prosthetic implant device of claim 12, wherein the wherein the proximal securement member and the second proximal securement member comprise one of a hook and loop enclosure or molded hooks.

14. The prosthetic implant device of claim 9, wherein the proximal end portion is closed when the first transverse portion is secured with the second transverse portion.

15. The prosthetic implant device of claim 9, wherein the first transverse portion defines a flap foldable over the second transverse portion.

16. A device configurable to facilitate insertion of a prosthetic implant into a patient, the device comprising:
a flexible sheet of sterilizable semitransparent or transparent material, the sheet comprising a proximal end portion, a distal end portion, a first side portion, and a second side portion;
the first side portion comprising a first side securement member, the first side securement member securable with the second side portion;
the proximal end portion comprising a first transverse portion having a proximal securement member, and a second transverse portion, the proximal securement member securable with the second transverse portion;
whereby to configure the device to facility insertion of the prosthetic implant, the proximal securement member of the first transverse portion is secured with the second transverse portion, the first side securement member of the first side portion is secured with the and the second side portion such that a distal opening in the distal end portion is formed.

17. The prosthetic implant device of claim 16, wherein the second side portion comprises a second side securement member adapted to secure with the side securement member of the first side portion, and the second side securement member comprises a plurality of attachment portions whereby the distal opening size is selected according to securing the side securement member with one of the plurality of attachment portions.

18. The prosthetic implant device of claim 16, wherein the second transverse portion comprises a second proximal securement member adapted to secure with the proximal securement member of the first transverse portion such that the proximal end portion is closed when the first transverse portion is secured with the second transverse portion.
